(19) United States
(12) Patent Application Publication
(10) Pub. No.: US 2020/0318176 A1
(43) Pub. Date: Oct. 8, 2020

(54) CONCENTRATION BASED DNA SEQUENCING MACHINE

(71) Applicant: Mohamed Mohamed Adel EL-SOKKARY, Mansoura (EG)

(72) Inventor: Mohamed Mohamed Adel EL-SOKKARY, Mansoura (EG)

(21) Appl. No.: 16/633,201

(22) PCT Filed: Jul. 25, 2018
(86) PCT No.: PCT/EG2018/000010

§ 371 Date: Jan. 23, 2020

(30) Foreign Application Priority Data
Jul. 26, 2017 (EG) 2017071237

(51) Int. Cl. C12Q 1/6869; B01L 3/00; B01L 7/00

(52) U.S. Cl. CPC C12Q 1/6869; B01L 3/502715; B01L 7/52; B01L 2300/18; C12Q 2537/165; C12Q 2565/607; C12Q 2537/157

(57) ABSTRACT

The term DNA sequencing is commonly applied to several methods and technologies that are used for determining the order of the nucleotide bases adenine, guanine, cytosine, and thymine in a molecule of DNA. It has many applications in numerous applied fields such as diagnostic, biotechnology, forensic biology and biological systematic, in the sequencing of the human genome, and in the Human Genome Project. In the presented machine, DNA sample fragments are amplified by usual PCR technique. The individual nucleotides are added to the nascent DNA. If the nucleotide is complementary to the tested DNA fragment, a change in the concentration of the added nucleotide could be traced. This change could be detected by any method indicating a complementary nucleotide. Finally, the combined data are used to generate sequence read-outs by computer system.

### TECHNICAL FIELD

This application is directed to DNA sequencing methodologies based on measuring the change in concentration of added nucleotide before and after the reaction

### BACKGROUND ART

Sequencing Principles:

Two main previously developed methodologies for DNA sequencing:

1—Sequencing by Synthesis:
Sanger/Dideoxy chain termination (Life Technologies, Applied Biosystems)
Pyrosequencing (Roche/454)
Reversible terminator (Illumina)
Zero Mode Waveguide (Pacific Biosciences) 3rd generation sequencing

2—Sequencing by Oligo Ligation Detection
SOLiD (Applied Biosystems)

The following is a more detailed description of these methods:

1—Chain Termination (Sanger) Sequencing:

In this method, a modified DNA replication reaction in which growing chains are terminated by dideoxynucleotides and the 3′-OH group necessary f′ formation of the phosphodiester bond is missing in ddNTPs. With the addition of enzyme (DNA polymerase), the primer is extended until a ddNTP is encountered. The chain will end with the incorporation of the ddNTP. With the proper dNTP:ddNTP ratio, the chain will terminate throughout the length of the template. All terminated chains will end in the ddNTP added to that reaction. The resulting terminated chains are resolved by electrophoresis. A distinct dye or “color” is used for each of the four ddNTP. Since the terminating nucleotides can be distinguished by color, all four reactions can be performed in a single tube.

2—Pyrosequencing:

Each nucleotide is added in turn in each cycle. Then only one of four will generates a light signal. For preparation to the next cycle, the remaining nucleotides are removed enzymatically. The light signal is recorded on a pyrogram

Pyrosequencing is based on the generation of light signal through release of pyrophosphate (PPi) on nucleotide addition.
DNAₙ+dNTP→DNAₙ₊₁+PP₁
PPi is used to generate ATP from adenosine phosphosulfate (APS).
APS+PP_{I}→ATP

ATP and luciferase generate light by conversion of luciferin to oxyluciferin

3—Zero Mode Waveguide:

This method is a single molecule real time sequencing based on the detection of the identity of each nucleotide immediately after its incorporation into a growing strand of DNA

4—Sequencing by Ligation:

Ligase instead of polymerase is used which joins probe sequences (DNA fragments instead of oligonucleotides). Once a probe is added, fluorescent signal is produced. Based on the fluorescence one can infer the identity of the nucleotide. Sequencing, method includes more than one primer that differs from the previous in only one base.

5—Nanopore Sequencing

Utilizes a nanoscale device that translocates polymer molecules in sequential monomer order through a very small volume of space. Includes a detector that directly converts characteristic features of the translocating polymer into an electrical signal. Transduction and recognition occur in real time, on a molecule-by-molecule basis. It can probe thousands of different molecules in a few minutes. It can probe very long lengths of DNA.

6—Sequencing-By-Synthesis (SBS):

SBS involves detection of the identity of each nucleotide immediately after its incorporation into a growing strand of DNA in a polymerase reaction. The SBS includes “fluorescent in situ sequencing” (FISSEQ) and the pyrosequencing method.

A different fluorophore is linked to each of the four bases through a photocleavable linker. DNA polymerase incorporates complementary a single-nucleotide analogue. Unique fluorescence emission detected depends upon the nt. incorporated. A fluorophore is subsequently removed photochemically and the 3-OH group is chemically regenerated and the cycle proceeds.

The Aim of this New in this Application:

Sequencing principles and sequencing machines includes tools and instruments highly expensive. In addition, it is not easy to obtain such technology in our labs. Most of these methods are difficult and requiring special enzymes or special labeling methods such as fluorescence dyes that increases the expenses of these experiments. For these reasons, new methods have to be developed and introduced to simplify this technology and decrease the cost associated.

### DISCLOSURE OF INVENTION

The New Principle:

The term DNA sequencing is commonly applied to several methods and technologies that are used for determining the order of the nucleotide bases adenine, guanine, cytosine, and thymine in a molecule of DNA. It has many applications in numerous applied fields such as diagnostic, biotechnology, forensic biology and biological systematic, in the sequencing of the human genome, and in the Human Genome Project. In the presented machine, DNA sample fragments are amplified by usual PCR technique. The individual nucleotides are added to the nascent DNA. If the nucleotide is complementary to the tested DNA fragment a change in the concentration of the added nucleotide which can be traced by any method

Detailed Description of the Device:

Genomic DNA fragments are fixed on a solid support. Each DNA fragment is amplified by PCR technique to produce a cluster of DNA. In the PCR technique, the temperature of the reaction mixture must be varied during a PCR cycle, from 95° C. to 40°-60° C., and finally to 72° C. for a certain number of cycles. Each cluster originated from a single DNA fragment acts as a single sequencing reaction. In the sequencing reaction, the DNA cluster is tested by adding one of the nucleotides at a time. If the nucleotide is complementary, a change of concentration of the added dNTP could be traced. This change could be tested directly in any part of the sequencing chamber.

Detailed Description of the Sequencing Chamber:

1. Solid support carrying the genomic DNA fragments to be amplified and then tested for complementary nucleotides. This chamber is equipped with electronic heating and cooling systems

2. Each DNA fragment is amplified by PCR technique to produce a cluster of DNA. In the PCR technique, the temperature of the reaction mixture must be varied during a PCR cycle, from 95° C. to 40°-60° C., and finally to 72° C. for a certain number of cycles

3. Four different solutions each carrying all the needed components to add one of the four types of nucleotides to the amplified DNA cluster are placed in four different containers.

4. In the sequencing reaction, the DNA cluster is tested by adding one of the nucleotides at a time. If the nucleotide is complementary, a change of concentration could be traced. This change could be tested directly in any part inside or outside the sequencing chamber.

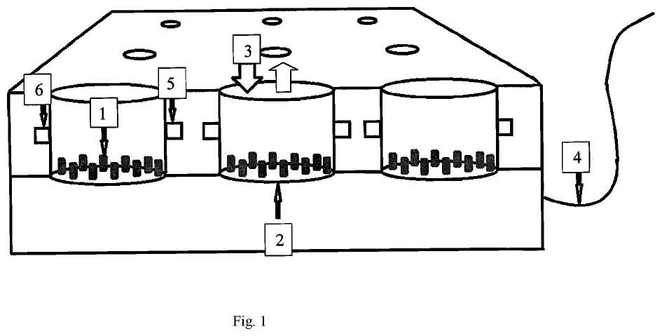

### BRIEF DESCRIPTION OF THE DRAWING: (FIG. 1)

1—Reaction chamber (1) containing solid support carrying the genomic DNA fragments to be amplified by any method

2—Heating and cooling system (2) of any type to control the temperature of the reaction chamber

3—Control unit for controlling all the processes carried out in the reaction chamber (4) connected to the input unit for software programming and the sensors (5),(6) inside or outside the reaction chamber that collect the data and send it to the control unit

4 Liquid and water transfer (3) system carrying all needed solutions to test the DNA cluster for complementary nucleotides by adding one of the nucleotides at a time and washing.

5—Measuring unit connected to the sensors (5) and (6) including all types of sensors needed to test if the nucleotide is complementary or not and to detect any change of concentration that could be traced. This change could be tested inside or outside the sequencing chamber. The data then is collected and can be send it to the main control unit.

1- A device for DNA sequencing comprising: a sample holder configured to receive a nucleic acid sample, a heating system configured to raise the temperature of the sample, a cooling system configured to lower the temperature of the sample, and a controller configured to control the heating system and the cooling system to cycle the device through a desired time-temperature profile. In addition, sensors of any type can be placed in any part inside or outside the reaction chamber providing information about the dNTP concentration before and after addition of each nucleotide using a suitable liquid and water transfer system.

2- The device of claim 1, wherein the sensors of any type can be placed in any part inside or outside the reaction chamber providing information about the state of the samples in the reaction chamber including: Temperature, and other factors.

3- The device of claim 1, wherein the control unit for controlling all the processes carried out in the reaction chamber connected to the input unit for software programming and the sensors inside and outside the reaction chamber that collect the data and send it to the control unit.

4- The device of claim 1, wherein the input unit is placed inside the machine for software programming and selection of the parameters.

5- The device of claim 1, wherein the temperature of the reaction chamber is controlled by any type of heating and cooling systems to control the temperature of the reaction chamber.

6- The device of claim 1, wherein many control samples can be placed inside the reaction chamber for more accuracy and reproducibility of the results.

7- The device of claim 1, wherein sensors of any type can be placed in any part inside or outside the reaction chamber providing information about the concentration before and after addition of each nucleotide.

8- The device of claim 1, wherein there is no specific number of samples.
